(19) United States
(12) Patent Application Publication
(10) Pub. No.: US 2013/0066324 A1
(43) Pub. Date: Mar. 14, 2013

(54) HYDRAULIC CEMENTS, METHODS AND PRODUCTS

(71) Applicants: Håkan Engqvist, Osthammar (SE); Jonas Aberg, Uppsala (SE)

(72) Inventors: Håkan Engqvist, Osthammar (SE); Jonas Aberg, Uppsala (SE)

(21) Appl. No.: 13/229,539

(22) Filed: Sep. 9, 2011

(51) Int. Cl. A61F 2/00; C09K 3/00; C04B 12/02

(52) U.S. Cl. 606/92; 106/691; 106/35

(57) ABSTRACT

A hydraulic cement composition comprises a mixture of (a) a cement powder composition which is soluble or partly soluble in water, (b) a non-aqueous water-miscible liquid, and (c) an aqueous hydration liquid. Methods of producing a hardened cement, hardened cements, kits, and articles of manufacture employ such compositions.

### FIELD OF THE INVENTION

The present invention is directed to hydraulic cements. The hydraulic cement compositions may be formed into hardened cements and, in a specific embodiment, the hydraulic cements are suitable for use as biomaterials for in vivo delivery, for example for bone and tooth restoration. The invention is also directed to hardened cements formed from such hydraulic cement compositions and to methods of producing hardened cements. The invention is further directed to kits and articles of manufacture including, inter alia, such hydraulic cement compositions.

### BACKGROUND OF THE INVENTION

Self-hardening calcium phosphate cements (CPC) have been used for bone and tooth restoration and for local drug delivery applications. See, for example, Larsson et al, “Use of injectable calcium phosphate cement for fracture fixation: A review,” Clinical Orthopedics and Related Research, 395:23-32 (2002) and Oda et al, “Clinical use of a newly developed calcium phosphate cement (XSB-671D),” Journal of Orthopedic Science, 11(2):167-174 (2006). The cements in powder form are typically mixed with an aqueous solution immediately before application. In the clinical situation, the ability of the surgeon to properly mix the cement powder and hydrating liquid and then place the cement paste in a defect within the prescribed time is a crucial factor in achieving optimum results. Specifically, the dry cement powder material needs to be mixed with an aqueous solution in the surgical setting, i.e., the operating room, transferred to an applicator, typically a syringe, and delivered to the desired location within the setting time. Conventional cements generally have a setting time of about 15-30 minutes. However, the methods used for mixing and transfer of cement for injection in the operating room are technically difficult and pose a risk for non-optimal material performance, e.g., early setting renders materials difficult to inject or causes phase separation, so called filter pressing. Further, for technical reasons and time constraints, the material is typically mixed with a hydrating liquid in bulk to form a paste and the paste is then transferred to smaller syringes for delivery. In practice, material is often wasted due to an early setting reaction, i.e., the hydrated material sets to a hardened cement prior to delivery to the desired location, or because excess material is mixed. A solution to these problems that includes the possibility to deliver material in smaller quantities in a more controlled manner is thus desired.

There are two common setting chemistries for CPCs which result in two different end products after setting, hydroxyapatite (also referred to hydroxylapatite) and Brushite. The apatite product results from a neutral to alkaline reaction, whereas the Brushite product results from an acidic reaction. Apatite cements generally have longer resorption time than an acidic cement. See, for example, Constantz et al, “Histological, chemical, and crystallographic analysis of four calcium phosphate cements in different rabbit osseous sites,” Journal of Biomedical Materials Research, 43(4):451-461 (1998). However, the long resorption time for apatite cements can pose a problem in a clinical setting where the cement is used for bone restoration. That is, it is preferable to have a cement resorption rate similar to the formation rate of new bone so that the regeneration of the bone is not inhibited. This is not the case for many apatite cements. See, for example, Miyamoto et al, “Tissue response to fast-setting calcium phosphate cement in bone,” Journal of Biomedical Materials Research, 37(4):457-464 (1997). It has been shown that biphasic cements combining larger granules of, for example, β-tricalcium phosphate (β-TCP) in a matrix of brushite or apatite cement or alternative cements in combination with bioglass, result in better biological responses, i.e., faster bone in-growth, than cements without such additives. Another method to improve the biological response of cements, e.g., to provide faster bone in-growth, is via addition of silicon, strontium and/or fluoride to the cement composition. See, for example, Guo et al, “The influence of Sr doses on the in vitro biocompatibility and in vivo degradability of single-phase Sr-incorporated HAP cement,” Journal of Biomedical Materials Research Part A, 86A(4):947-958 (2008) and Camire et al, “Material characterization and in vivo behavior of silicon substituted alpha-tricalcium phosphate cement,” Journal of Biomedical Materials Research Part B-Applied Biomaterials, 76B(2):424-431 (2006). On the other hand, the acidic Brushite cements are difficult to use in a clinical setting due to their rapid setting reaction, involving the disadvantages discussed above.

In addition, injectable self-hardening biomaterials based on calcium silicates have been proposed for use in bone repair in orthopedics (see US 2006/0078590) and endodontics (see WO 94/24955). These self-hardening cements based on calcium silicates are similarly formed by mixing of powder and liquid to form a paste. However, the mixing procedure is often performed using a spatula or via a mechanical mixing system. Non-homogeneous mixing and the formation of air voids in the cement paste often result. Non-homogeneous mixed cement and/or air voids result in low mechanical strength and difficulties in delivering the cement through thin needles without obtaining phase separation between liquid and powder (the filter pressing effect). Moreover, these cements are fast setting and typically, in practice, the rheology of the cement can increase to such an extent that complete delivery by injection is impossible.

Self-hardening cements based on calcium aluminate cements have also been proposed to be used as biomaterial (see US 2008/0210125). The calcium aluminate cement materials have a beneficial mechanical strength profile compared to calcium phosphate cements, and in addition, the calcium aluminate materials are considered to be non-resorbable. However, due to the anhydrous nature of the calcium aluminate powders and their rapid hardening behavior, it is difficult to obtain a combined long shelf life and easy mixing to achieve optimal clinical results.

The problem of obtaining a proper mix of the powder material and hydrating liquid for optimum clinical results in apatite cements has been addressed in US 2006/0263443, US 2007/0092856, Carey et al, “Premixed rapid-setting calcium phosphate composites for bone repair,” Biomaterials, 26(24):5002-5014 (2005), Takagi et al, “Premixed calcium-phosphate cement pastes,” Journal of Biomedical Materials Research Part B-Applied Biomaterials, 67B(2):689-696 (2003), Xu et al, “Premixed macroporous calcium phosphate cement scaffold,” Journal of Materials Science-Materials in Medicine, 18(7):1345-1353 (2007), and Xu et al, “Premixed calcium phosphate cements: Synthesis, physical properties, and cell cytotoxicity,” Dental Materials, 23(4):433-441 (2007), wherein premixed pastes are described. In US 2006/0263443, for example, a powder composition for hydroxyapatite is premixed with an organic acid and glycerol to form a paste, which paste may subsequently be injected into a defect. The injected material hardens via the diffusion of body liquids into the biomaterial. The organic acid is added to increase resistance to washout and the end product after setting is apatite, which is known to have a long resorption time in vivo as described above. Also, compositions of β-tricalcium phosphate (β-TCP) and hydrated acid calcium phosphate in glycerin or polyethylene glycol have previously been described in CN 1919357. Han et al, “β-TCP/MCPM-based premixed calcium phosphate cements,” Acta Biomaterialia, doi:10.1016/j.actbio.2009.04.024 (2009), also discloses premixed cements.

However, there is a continuing need to be able to efficiently prepare and safely deliver hydraulic cements, particularly for biomedical applications, i.e., hydraulic cements that overcome the above noted and/or other difficulties of conventional hydraulic cement materials, while optionally optimizing performance properties.

### SUMMARY OF THE INVENTION

Accordingly, it is an object of the present invention to provide hydraulic cements, hardened cements, and methods, kits and articles of manufacture based on the hydraulic cements. By using a liquid that is a mixture of a non-aqueous water-miscible liquid and an aqueous hydration liquid, i.e., water, the setting time of the precursor powder is considerably lowered while the working time prior to setting is sufficiently long to allow delivery of the cement, and the hardened cement has high mechanical strength. The present cements are therefore advantageous as compared with cements mixed with only water-based liquids, which exhibit similar strength and setting time but very limited working time compared to present invention, and cements mixed with only glycerol-based liquids, which exhibit similar strength and working time, but longer setting time compared to present invention.

In one embodiment, the invention is directed to a hydraulic cement composition which comprises a mixture of (a) a cement powder composition which is soluble or partly soluble in water, (b) a non-aqueous water-miscible liquid, and (c) a hydration liquid. The cement powder composition contains at least one nonhydrated cement powder and, in addition, may optionally contain hydrated powders, filler particles, and the like which do not participate in a cement forming hydration reaction.

In one specific embodiment, the invention is directed to a hydraulic cement composition which comprises a mixture of (a) a Brushite or Monetite-forming calcium phosphate powder composition, (b) non-aqueous water-miscible liquid, and (c) a hydration liquid. For clarification, an example of a non-aqueous water-miscible liquid includes glycerol, an almost water free liquid that can be dissolved in water.

In another specific embodiment, the invention is directed to a hydraulic cement composition which comprises a mixture of (a) a non-hydrated powder composition comprising porous β-tricalcium phosphate (β-TCP) granules and at least one additional calcium phosphate powder, (b) non-aqueous water-miscible liquid, and (c) a hydration liquid.

In another specific embodiment, the invention is directed to a hydraulic cement composition which comprises a mixture of (a) a non-hydrated powder composition comprising calcium silicate powder (b) non-aqueous water-miscible liquid, and (c) a hydration liquid.

In a further specific embodiment, the invention is directed to a hydraulic cement composition which comprises a mixture of (a) a non-hydrated powder composition comprising calcium aluminate powder, (b) non-aqueous water-miscible liquid, and (c) a hydration liquid.

The invention is also directed to methods of producing a hardened cement with such compositions, which methods comprise contacting a hydraulic cement premix composition with an aqueous hydration liquid, wherein the hydraulic cement premix composition comprises a) a cement powder composition which is soluble or partly soluble in water, and (b) a non-aqueous water-miscible liquid. The invention is also directed to hardened cements produced from such compositions, kits for providing such compositions, and articles of manufacture for providing such compositions.

The hydraulic cement compositions according to the invention are advantageous in that they avoid many of the point of use preparation difficulties of conventional hydraulic cement compositions, particularly when used as biomaterials, and may be easily and efficiently delivered to a desired location, without excessive material waste. Additionally, the hydraulic cement compositions according to the invention may be optimized for improved performance properties, such as injectability, setting time and strength. These and additional objects and advantages of the present invention will be more fully appreciated in view of the following detailed description.

### DETAILED DESCRIPTION

The hydraulic cement compositions of the present invention are suitable for use in various applications. The present description refers to use of the compositions for in vivo applications, for example in bone and tooth repair. It will be appreciated that the present compositions are suitable for other in vivo applications as well as for non-biomaterial applications. The compositions of the invention employ a premix of a non-hydrated powder and non-aqueous water miscible liquid which hydrates and forms a set cohesive cement upon contact with a hydrating liquid or vapor, typically water or an aqueous solution.

The inclusion of a hydration liquid in the hydraulic cement compositions of the present invention improves the mechanical properties of the set cement material. Furthermore, the hydration liquid makes the cement less viscous, thus improving the injectability. Additionally the hydration liquid reduces the setting time of the hydraulic cement composition. The amount of hydration liquid is chosen so that the cement does not set prematurely, i.e. the cement has a constant low viscosity for an extended time, thus giving, for example, a surgeon sufficient time for application of the cement.

In a first embodiment, the hydraulic cement composition comprises a mixture of (a) a Brushite or Monetite-forming calcium phosphate powder composition, (b) non-aqueous water-miscible liquid, and (c) a hydration liquid. In order to be Brushite-forming or Monetite-forming, the calcium phosphate powder composition is acidic, i.e., the pH of the hydraulic cement composition during setting is less than about 6.0. Thus, in a broad embodiment, the calcium phosphate powder is acidic and an acidic cement is formed. In a specific embodiment, the Brushite or Monetite-forming calcium phosphate powder composition comprises an acidic phosphate, for example, monocalcium phosphate monohydrate (MCPM), anhydrous monocalcium phosphate (MCPA), phosphoric acid, pyrophosphoric acid, or a mixture thereof. In a more specific embodiment, the Brushite or Monetite-forming powder composition comprises monocalcium phosphate monohydrate, anhydrous monocalcium phosphate, or a mixture thereof. The Brushite or Monetite-forming powder composition may further comprise one or more basic calcium phosphates, as long as the pH of the hydraulic cement composition during setting is less than about 6.0 and the result is a Brushite or Monetite cement. Thus, the Brushite or Monetite-forming calcium phosphate powder composition may further comprise one or more calcium phosphates selected from the group consisting of anhydrous dicalcium phosphate, dicalcium phosphate dihydrate, octacalcium phosphate, α-tricalcium phosphate, β-tricalcium phosphate, amorphous calcium phosphate, calcium-deficient hydroxyapatite, non-stoichiometric hydroxyapatite, and tetracalcium phosphate.

In specific embodiments, the MCPA and or MCPM particle size is preferably less than about 400 μm, more preferably less than about 200 μm and most preferably less than about 100 μm, the particle size distribution of the α-tricalcium phosphate, β-tricalcium phosphate, and/or tetracalcium phosphate is preferably characterized by a dv 0.5 of about 1 to 40 μm, more specifically about 3 to 30 um and even more specifically about 5 to 25 μm, and the P/L is about 2-7, more specifically about 3-6 for a cement with higher mechanical strength.

In a second embodiment, the hydraulic cement composition comprises a mixture of (a) a non-hydrated powder composition comprising porous β-tricalcium phosphate (β-TCP) granules and at least one additional calcium phosphate powder, (b) non-aqueous water-miscible liquid, and (c) a hydration liquid. The porous β-TCP granules modify the resorption rate and bone remodelling of the hardened cement which is formed upon hydration and setting. The granules generally comprise agglomerated powders and the porosity of the granules comprises pores formed between individual powder grains in the agglomerates. In a specific embodiment, the granule size is from about 10 to about 3000 micrometers. In a further embodiment, the granule size is from about 10 to about 1000 micrometers and may be selected to optimize mechanical and/or biological properties of the resulting hardened cement. In a specific embodiment, the granule porosity is at most 80 vol % and the pore size is at most about 500 micrometers, or, more specifically, at most about 200 micrometers.

In a specific embodiment, the weight ratio of porous β-TCP granules to additional calcium phosphate powder in the non-hydrated powder composition is in a range about 1:20 to about 1:1, or, more specifically, in a range of about 1:9 to about 1:2. The additional calcium phosphate powder may comprise an acidic powder, a basic powder, or a mixture thereof. In one embodiment, the additional calcium phosphate powder comprises one or more of monocalcium phosphate monohydrate (MCPM), monocalcium phosphate anhydrous (MCPA), dicalcium phosphate anhydrous (DCPA), dicalcium phosphate dihydrate (DCPD), octacalcium phosphate (OCP), α-tricalcium phosphate (α-TCP), β-tricalcium phosphate (β-TCP), amorphous calcium phosphate, calcium-deficient hydroxyapatite (HA), non-stoichiometric HA, ion-substituted HA, and tetracalcium phosphate (TTCP). In a more specific embodiment, the additional calcium phosphate powder comprises an acidic powder and, more specifically, monocalcium phosphate monohydrate (MCPM), anhydrous monocalcium phosphate (MCPA), or a mixture thereof. In yet a further embodiment, the additional calcium phosphate powder comprises an acidic powder, for example, monocalcium phosphate monohydrate (MCPM), anhydrous monocalcium phosphate (MCPA), or a mixture thereof, and a basic powder, for example, tetracalcium phosphate, octacalcium phosphate (OCP), α-tricalcium phosphate (α-TCP), β-tricalcium phosphate (β-TCP), amorphous calcium phosphate, calcium-deficient HA, non-stoichiometric HA, ion-substituted HA, tetracalcium phosphate (TTCP) or combinations thereof. In a specific embodiment, the basic powder constitutes at least 30 wt. % of the powder composition. The components of the powder compositions are chosen in such an amount that either (i) the pH of the cement paste during setting is lower than 6, or (ii) the pH of the cement paste during setting is above 6, or (iii) a combination of (i) and (ii) with an first initial pH below 6 followed by a pH above 6 during the setting reaction, or (iv) a first neutral pH followed by a pH below 6 during the setting reaction. Depending on the pH of the powder composition during setting of the cement material, the end-product may comprise amorphous calcium phosphate hydrate, hydroxyapatite, ion-substituted hydroxyapatite, dicalcium phosphate dihydrate (brushite) or Ca(HPO₄) (monetite), or combinations thereof.

According to one specific embodiment, the powder composition is acidic and comprises (a) a basic calcium phosphate component comprising the porous β-TCP granules and optionally tetra calcium phosphate (TTCP) and/or amorphous calcium phosphate, and (b) an acidic phosphate, non-limiting examples of which include monocalcium phosphate monohydrate (MCPM), anhydrous monocalcium phosphate, phosphoric acid, pyrophosphoric acid or combinations thereof. The weight ratio between components (a) and (b) may be in the range of about 3:1 to about 1:3. The components of the powder composition are chosen such that (i) the pH of the cement paste during setting is lower than 6.0; and (ii) the end-product of the setting reaction comprises dicalcium phosphate dihydrate (brushite) or Ca(HPO₄) (monetite) or a combination thereof.

In an alternate embodiment, the powder composition is basic (apatitic) and comprises (a) a basic calcium phosphate component comprising the porous β-TCP granules and optionally tetra calcium phosphate (TTCP) and/or amorphous calcium phosphate, and (b) an acidic phosphate, non-limiting examples of which include monocalcium phosphate monohydrate (MCPM), anhydrous monocalcium phosphate, phosphoric acid, pyrophosphoric acid or combinations thereof. The components of the apatitic powder compositions are chosen such that (i) the pH of the cement paste during setting is higher then 6; and (ii) the end-product of the setting reaction comprises amorphous calcium phosphate hydrate, hydroxyapatite, ion-substituted hydroxyapatite, or combinations thereof.

In a third embodiment of the invention, the hydraulic cement composition comprises a mixture of (a) a non-hydrated powder composition comprising calcium silicate powder, (b) non-aqueous water-miscible liquid (c) a hydration liquid. When hydrated, the composition forms mainly a calcium silicate hydrate. In a specific embodiment, the powder composition comprises 20-100 weight % calcium silicate, for example, CaOSiO₂, (CaO)₃SiO₂, and/or (CaO)₂SiO₂. In one embodiment, to optimize a clinically acceptable setting time, the composition includes (CaO)₃SiO₂ or (CaO)₂SiO₂ or combinations thereof, or, more specifically, (CaO)₃SiO₂. It is often difficult to obtain a 100% pure phase composition and therefore trace amounts of all calcium silicate phases may be present in the composition. The grain size of the calcium silicate powder is generally below 200 micrometer, preferably below 50 micrometer. This to obtain an optimal combination of injectability (coarse powder) and strength (fine grain size).

In a fourth embodiment, the invention is directed to a hydraulic cement composition comprising a mixture of (a) a non-hydrated powder composition comprising calcium aluminate powder, (b) non-aqueous water-miscible liquid (c) a hydration liquid. The calcium aluminate powder comprises one or more powders selected from the group consisting of (CaO)₃Al₂O₃, (CaO)₁₂(Al₂O₃)₇, (CaO)Al₂O₃, CaO(Al₂O₃)₂, and CaO(Al₂O₃)₆. In a specific embodiment, wherein the setting time may be optimized, the calcium aluminate powder comprises one or more powders selected from the group consisting of (CaO)₃Al₂O₃, (CaO)₁₂(Al₂O₃)₇, and (CaO)Al₂O₃. In a more specific embodiment, the calcium aluminate powder comprises (CaO)₁₂(Al₂O₃)₇ and/or (CaO)Al₂O₃ and in a more specific embodiment, the calcium aluminate powder comprises (CaO)Al₂O₃. In one embodiment, the calcium aluminate is amorphous, more specifically amorphous (CaO)₁₂(Al₂O₃)₇. Upon hydration, a hardened cement comprising calcium aluminate hydrate is formed. The grain size of the calcium silicate powder is generally below 200 micrometer, preferably below 50 micrometer. This to obtain an optimal combination of injectability (coarse powder) and strength (fine grain size).

In a specific embodiment, the powder composition comprises at least about 10 weight %, or from about 10 to about 100 weight %, of calcium aluminate powder. In a more specific embodiment, the powder composition comprises at least about 50 weight percent of the calcium aluminate powder to provide high strength. In a further embodiment, the powder composition comprises from about 3 to about 60 weight %, specifically from about 3 to about 50 weight %, more specifically from about 10 to about 30 weight %, of an agent operable to increase radio-opacity of the composition. Examples of such agents include, but are not limited to, zirconium dioxide, barium sulfate, iodine and strontium compounds and combinations thereof. The increased radio-opacity provided by such an agent is important to increase safety during injection (high visibility compared to bone tissue) and follow up when set in vivo. The powder composition may also optionally include microcrystalline silica which may be added to control expansion properties of the material. In one embodiment, the powder composition comprises from about 0.1 to about 15 weight %, more specifically from about 0.1 to about 5 weight %, of microcrystalline silica.

In a fifth embodiment, the invention is directed to a hydraulic cement composition comprising a mixture of (a) a non-hydrated powder composition comprising calcium sulphate powder, (b) non-aqueous water-miscible liquid (c) a hydration liquid. The calcium sulfate powder may be of the dihydrate, hemihydrate (alfa or beta or combinations thereof), and/or anhydrate structures. In one embodiment, calcium sulfate of the alpha-hemihydrate structure is preferred owing to its higher strength and lower rapid setting time. The particle size distribution of the calcium sulphate powder is preferably <100 μm and more preferably <50 μm. This to obtain an optimal combination of injectability (coarse powder) and strength (fine grain size).

The powder to liquid (i.e., non-aqueous water-miscible liquid and hydration liquid) weight to volume ratio (P/L ratio) may suitably be in a range of from about 0.5 to about 10, more specifically from about 1 to about 7, and more specifically from about 2.5 to about 7, or from about 2.5 to about 6, for better handling and mechanical strength. These ratios are suitable even if two or more non-aqueous water-miscible liquids and/or hydration liquids are used in combination.

Any suitable, non-aqueous water-miscible liquid may be employed. Exemplary liquids include, but are not limited to, glycerol, propylene glycol, poly(propylene glycol), poly(ethylene glycol) and combinations thereof, and related liquid compounds and derivatives, i.e., substances derived from non-aqueous water miscible substances, substitutes, i.e., substances where part of the chemical structure has been substituted with another chemical structure, and the like. Certain alcohols may also be suitable. In a specific embodiment, the liquid is glycerol.

Any suitable hydrating liquid is employed. The hydration liquid may be any polar liquid, such as water or polar protic solvents (e.g. alcohol). The hydrating liquid is suitably water or an aqueous solution. The hydration liquid can optionally have a pH within the range of 1-9.

The concentration of the hydration liquid, based on the combination of the hydration liquid and the non aqueous water miscible liquid combined, may suitably be in a range of 1 to 50% (v/v), more specifically from 2-40%, and more specifically from 3-30% for better mechanical strength and adequate handling properties.

The compositions may also include one or more porogens to give a macroporous end product to facilitate fast resorption and tissue in-growth. The pores give a good foundation for bone cells to grow in. The porogen may include sugars and other fast-resorbing agents, and non-limiting examples include calcium sulphate, mannitol, poly(a-hydroxy ester) foams, sucrose, NaHCO₃, NaCl and sorbitol. The amount of porogen may suitably be from about 5 to about 30 weight % of the powder composition. The grain size of the porogens are typically in the range of 50 to 600 μm.

The hydraulic cement compositions in the form of a premixed paste may be delivered, for example to an implant site when used as a biomaterial, using a syringe or spatula. The hydraulic cement compositions may be shaped in vivo, and subsequently be hydrated or be allowed to hydrate in vivo. Optionally, a water-containing liquid can be added to the premixed paste just before delivery in the operating room, for example, into a jar.

The hydraulic cement compositions in the form of a premixed paste can also be packaged in a vacuum package to reduce the amount of air voids in the paste and thus increase the final strength of the hardened material. Air voids reduce the strength of the set material and reduction of air voids is therefore important. The hydraulic cement compositions may be conveniently mixed and packaged under vacuum conditions. Preferably the hydraulic cement compositions are vacuum-mixed (e.g. in a Ross Vacuum Mixer Homogenizer).

In one embodiment, a premix is formed of the cement composition components other than the aqueous hydration liquid. The hardened cement is then formed by contacting the premix with the aqueous hydration liquid and allowing the resulting mixture to set. The aqueous hydration liquid may be added to the premix, for example, by mixing prior to delivery of the cement composition to an environment of use. Alternatively, the aqueous hydration liquid may comprise a body fluid, i.e., saliva, blood or the like, which is contacted with the premix once the premix is delivered in vivo. Alternatively, the aqueous hydration liquid may be provided in the form of an aqueous bath, which is suitable, for example, for molding complex shapes with subsequent hardening in water-containing bath. The hardening can optionally be performed at elevated temperatures, i.e., greater than about 25° C., up to, for example, about 120° C., for faster hardening and can also be used to control the phase of the hardened material. Such hardened materials can for example be used as custom made implants or for implants with a complex geometry difficult to achieve via normal powder processing routes.

In another embodiment of the invention, the hydraulic cement compositions, or the premix thereof which omits the aqueous hydration liquid, may be provided as an article of manufacture and/or a component of a kit, for example in combination with a separately contained quantity of hydration liquid. In a specific embodiment, the kit comprises several prefilled syringes of the same or of various sizes. One non-limiting example is a kit with several 2 ml prefilled syringes. Another non-limiting example is a kit with several 1 ml prefilled syringes. Thus, another embodiment of the invention comprises an article of manufacture comprising a hydraulic cement composition in a dispensing container, more specifically a syringe.

In one embodiment, an article of manufacture comprises a first container containing a hydraulic cement premix composition comprising (a) a cement powder composition which is soluble or partly soluble in water, and (b) a non-aqueous water-miscible liquid, and a second container containing a quantity of aqueous hydration liquid. In a specific embodiment, the first container and the second container may be in the form of a double barrel syringe. Suitably, such a syringe may additionally provide for mixing of the premix and aqueous hydration liquid prior to of upon dispensing. In another embodiment, the first container is a vacuum package. Suitably, the quantity of aqueous hydration liquid comprises about 1-50 volume percent of the combined volume of the non-aqueous water-miscible liquid and the aqueous hydration liquid.

The described hydraulic cement compositions are suitably employed as injectable in situ-setting biomaterials. The compositions can be used as any implant, more specifically as a bone implant, more specifically as dental or orthopedic implant. In a specific embodiment, the hydraulic cement compositions are suitable used as material in cranio maxillofacial defects (CMF), bone void filler, trauma, spinal, endodontic, intervertebral disc replacement and percutaneous vertebroplasty (vertebral compression fracture) applications.

Various embodiments of the invention are illustrated in the following Examples.

### Example 1

This example demonstrates the effect of adding a hydration liquid such as water to a premixed cement formulation. The addition of 5-15% water increases the compressive strength significantly and also decreases the injection force and the setting time.

### Cement Preparation

A first type of cement consisted of monocalcium phosphate anhydrous (MCPA, grain size below 600 micrometer) and β-tri calcium phosphate (β-TCP, Sigma, grain size below 40 micrometer), in a molar ratio of 1:1. Glycerol (anhydrous) was used as a mixing liquid with a water concentration of 0, 7.5, 15, 22.5 and 30% (v/v). The powder to glycerol ratio was 4 (g/mL). A vacuum mixer was used to mix the cements. The MCPA was obtained by heating monocalcium phosphate hydrate (Alfa Aesar) to 110° C. for 24 hours.

A second type of cement consisted of calcium trisilicate (CaO)₃SiO₂ (C3S, grain size below 30 micrometer) and (β-TCP, Sigma, grain size below 40 micrometer) and CaCl₂, in a molar ratio of 5:1:0.1. Glycerol (anhydrous) was used as mixing liquid with a water concentration of 0 and 30% (v/v). The powder to liquid ratio was 4 (g/mL). A vacuum mixer was used to mix the cements. The injectability was not studied for the cement.

A third type of cement consisted of calcium monoaluminate CaOAl₂O₃ (CA, grain size below 30 micrometer), Zirconia, grain size below 40 micrometer, LiCl and microsilica in a molar ratio of 4:1:0.1:0.5. Glycerol (anhydrous) was used as mixing liquid with a water concentration of 0 and 30% (v/v). The powder to liquid ratio was 4 (g/mL). A vacuum mixer was used to mix the cements. The injectability was not studied for the cement.

### Injectability

The injectability was evaluated by measuring the force needed to inject 2 ml of cement paste from a disposable syringe; barrel diameter 8.55 mm, outlet diameter 1.90 mm. The force applied to the syringe during the injection was measured and mean injection force from 10 to 30 mm displacement was calculated, this force is referred to as the injection force.

### Setting Time (ST)

To evaluate setting time of the cement, the cement was injected in four cylindrical moulds diameter 6 mm, height 3 mm. At t=0, the filled moulds were immersed in 37° C. phosphate buffered saline solution (PBS, pH 7.4, Sigma), to simulate in vivo conditions. The cement was considered to have set when the sample could support the 453.5 g Gillmore needle with a tip diameter of 1.06 mm without breaking.

### Compressive Strength (CS)

For CS measurements, the paste was injected into cylindrical moulds and immersed in 50 ml PBS at 37° C. in a sealed beaker. Sample dimensions were diameter 6 mm and height 12 mm. After 24 h, the samples were removed from the moulds and carefully polished to obtain the correct height and parallel surfaces. The maximum compressive stress until failure was measured.

The results are set forth in Tables 1-3:

TABLE 1
Calcium phosphate cement

| Water | Injection | Setting time | Compressive |
|---|---|---|---|
| (%) | force (N) | (min) | strength (MPa) |
| 0 | 110 | 30-35 | 6-8 |
| 7.5 | 35 | 15-20 | 10-13 |
| 15 | 15 | 10-15 | 10-14 |
| 22.5 | 15 | 9-12 | 8-10 |
| 30 | 10 | 4-8 | 5-7 |

TABLE 2
Calcium silicate cement

| Water | Setting time | Compressive |
|---|---|---|
| (%) | (min) | strength (MPa) |
| 0 | >240 | n.d. (too long |
| | | setting time) |
| 30 | <120 | 50 |

TABLE 3
Calcium aluminate cement

| Water | Setting time | Compressive |
|---|---|---|
| (%) | (min) | strength (MPa) |
| 0 | ~120 | 60 |
| 30 | <30 | 80 |

The results shows that the addition of a hydration liquid such as water it is possible to increase the strength at the same time as the setting time is reduced. The injectability of the cements were not studied closely however the viscosity of the cements containing water were less viscous then the non-aqueous mixtures and easier to inject into the sample moulds.

### Example 2

A series of experiments were performed to study the influence of hardening temperature on the mechanical properties of the cements.

### Cement Formulation

The cement consisted of monocalcium phosphate anhydrous (MCPA) and β-tri calcium phosphate (β-TCP, Degradeble Solutions), in a molar ratio of 1:1. Glycerol (anhydrous) was used as mixing liquid with a water concentration of 0, 7.5, 15, 22.5 and 30% (v/v). The powder to liquid ratio was 4 (g/mL). A vacuum mixer was used to mix the cements. The MCPA was obtained by heating monocalcium phosphate hydrate (MCPM, Alfa Aesar) to 110° C. for 24 hours.

### Compressive Strength (CS)

For CS measurements, the paste was injected into cylindrical moulds and immersed in 50 ml PBS at 37° C. in a sealed beaker. Sample dimensions were diameter 6 mm and height 12 mm. After 24 h, the samples were removed from the moulds and carefully polished to obtain the correct height and parallel surfaces. The maximum compressive stress until failure was measured. The results are set forth in Table 4:

TABLE 4
Results

| | Water | Compressive |
|---|---|---|
| P/L | (%) | strength (MPa) |
| 4.1 | 18 | 27¹, 29³ |
| 4.3 | 19 | 23¹, 26², |

¹Hardened at 37° C.
²Hardened at 60° C.
³Hardened at 90° C.

The results showed that an increase in compressive strength is obtained for higher hardening temperatures. For the embodiments that include forming an implant and providing it in hardened state in vivo, an increase in hardening temperature gives a stronger product. Also, it was noted that the hardening is faster for higher hardening temperatures.

The specific examples and embodiments described herein are exemplary only in nature and are not intended to be limiting of the invention defined by the claims. Further embodiments and examples, and advantages thereof, will be apparent to one of ordinary skill in the art in view of this specification and are within the scope of the claimed invention.

What is claimed is:

1. A hydraulic cement composition, comprising a mixture of (a) a cement powder composition which is soluble or partly soluble in water, (b) a non-aqueous water-miscible liquid, and (c) an aqueous hydration liquid.

2. The hydraulic cement composition of claim 1, comprising about 1-50 volume percent of the aqueous hydration liquid, based on the combined volume of (b) the non-aqueous water-miscible liquid, and (c) the aqueous hydration liquid.

3. The hydraulic cement composition of claim 1, comprising about 3-30 volume percent of the aqueous hydration liquid, based on the combined volume of (b) the non-aqueous water-miscible liquid, and (c) the aqueous hydration liquid.

4. The hydraulic cement composition of claim 1, wherein the non-aqueous water-miscible liquid comprises glycerol and the aqueous hydration liquid is water.

5. The hydraulic cement composition of claim 1, wherein the ratio of the (a) cement powder composition (weight) to (b) the non-aqueous water-miscible liquid and (c) the aqueous hydration liquid (volume) (P/L ratio) is about 0.5-10.

6. The hydraulic cement composition of claim 1, wherein the cement powder composition comprises a Brushite or Monetite-forming calcium phosphate powder composition.

7. The hydraulic cement composition of claim 6, wherein the Brushite or Monetite-forming calcium phosphate powder composition comprises monocalcium phosphate monohydrate, anhydrous monocalcium phosphate, or a mixture thereof.

8. The hydraulic cement composition of claim 1, wherein the cement powder composition comprises porous β-tricalcium phosphate (β-TCP) granules and at least one additional calcium phosphate powder.

9. The hydraulic cement composition of claim 8, wherein the at least one additional calcium phosphate powder comprises monocalcium phosphate monohydrate, anhydrous monocalcium phosphate, or a mixture thereof.

10. The hydraulic cement composition of claim 9, wherein the at least one additional calcium phosphate powder further comprises a basic powder comprising tetracalcium phosphate, octacalcium phosphate (OCP), α-tricalcium phosphate (α-TCP), amorphous calcium phosphate, calcium-deficient hydroxyapatite (HA), non-stoichiometric HA, ion-substituted HA, tetracalcium phosphate (TTCP) or combinations thereof.

11. The hydraulic cement composition of claim 1, wherein the cement powder composition comprises calcium silicate powder.

12. The hydraulic cement composition of claim 1, wherein the cement powder composition comprises a non-hydrated powder composition comprising calcium aluminate powder.

13. A method of preparing a hardened cement, comprising contacting a hydraulic cement premix composition with an aqueous hydration liquid, wherein the hydraulic cement premix composition comprises a) a cement powder composition which is soluble or partly soluble in water, and (b) a non-aqueous water-miscible liquid.

14. The method of claim 13, wherein the aqueous hydration liquid comprises water.

15. A hardened cement formed according to the method of claim 13.

16. The method of claim 13, wherein the non-aqueous hydraulic cement composition is injected in vivo and the aqueous hydration liquid comprises a body fluid.

17. An article of manufacture, comprising a first container containing a hydraulic cement premix composition comprising (a) a cement powder composition which is soluble or partly soluble in water, and (b) a non-aqueous water-miscible liquid, and a second container containing a quantity of aqueous hydration liquid.

18. The article of manufacture of claim 17, wherein the first container and the second container form a double barrel syringe.

19. The article of manufacture of claim 17, wherein the first container is a vacuum package.

20. The article of manufacture of claim 17, wherein the quantity of aqueous hydration liquid comprises about 1-50 volume percent of the combined volume of the non-aqueous water-miscible liquid, and the aqueous hydration liquid.
